(19) United States
(12) Patent Application Publication
(10) Pub. No.: US 2019/0180481 A1
(43) Pub. Date: Jun. 13, 2019

(54) TOMOGRAPHIC RECONSTRUCTION WITH WEIGHTS

(71) Applicants: General Electric Company, Schenectady, NY (US); Purdue University, West Lafayette, IN (US); Notre Dame University, Notre Dame, IN (US)

(72) Inventors: Lin Fu, Niskayuna, NY (US); Jean-Baptiste Thibault, Waukesha, WI (US); Somesh Srivastava, Waukesha, WI (US); Charles A. Bouman, Jr., West Lafayette, IN (US); Donghye Ye, West Lafayette, IN (US); Amirkoushyar Ziabari, West Lafayette, IN (US); Ken Sauer, Notre Dame, IN (US)

(21) Appl. No.: 15/840,953

(22) Filed: Dec. 13, 2017

(51) Int. Cl. G06T 11/00; A61B 6/03

(52) U.S. Cl. CPC G06T 11/006; A61B 6/032; G06T 2207/10076; G06T 2207/20; G06T 2207/10081; G06T 2211/424

(57) ABSTRACT

An iterative reconstruction approach is provided that allows the use of differing weights in pixels or larger sub-regions in the reconstructed image. By way of example, the relative significance of each projection measurement may be determined based on both the measurement position and the location of the reconstructed pixel. Computationally, the significance of each projection based on these two factors is represented by a weight factor employed in the algorithmic computation.

### BACKGROUND

Non-invasive imaging technologies allow images of the internal structures or features of a patient to be obtained without performing an invasive procedure on the patient. In particular, such non-invasive imaging technologies rely on various physical principles (such as the differential transmission of X-rays through the target volume, the reflection of acoustic waves within the volume, the paramagnetic properties of different tissues and materials within the volume) to acquire data and to construct images or otherwise represent the observed internal features of the patient.

In some imaging techniques such as computed tomography (CT), positron emission tomography (PET), single photon emission tomography (SPECT), magnetic resonance imaging (MM), etc., it may be desirable to employ an iterative reconstruction approach, as opposed to direct or analytic reconstruction approaches, to reconstructing the images. Such iterative approaches are computationally intensive and time-consuming but may allow data to be acquired at a lower dose, relying on the iterative processing to provide images of a useful quality.

However, such iterative approaches have limitations in addition to their computational intensity. For example, conventional CT iterative tomographic reconstruction (IR) uses statistical weights to modulate the importance of each sinogram ray for benefits in dose efficiency and image quality. However, because these weights are assigned in the sinogram-domain ray-by-ray, they apply globally to all image locations along the full length of the ray. Similarly, in IR methods for other imaging technologies such as PET, SPECT, MRI, and so forth, the weight assigned to each measured value applies globally to all image locations contributing to that measurement.

While this provides the radiation dose and image quality benefits noted, it prevents differential treatment of sub-regions within the image, such as adaptive weighting of the measurements for individual pixel locations or sub-regions of the image. This may run counter to the needs of a given examination, where in many instances individual sub-regions in the image may benefit from different treatment of the same measurement to achieve the best image quality.

### BRIEF DESCRIPTION

In one aspect of the present approach, a tomographic iterative reconstruction method is provided. In accordance with this embodiment, a set of projection data of a scanned region is accessed or acquired. A reconstruction operation is iteratively performed to reconstruct an image of the region. As part of each reconstruction operation, a weight factor is applied to each projection measurement. The respective weight factors are determined based on both a respective projection measurement position and a reconstructed pixel location. The image is displayed or stored.

In a further aspect of the present approach, an image reconstruction system is provided. In accordance with this aspect, the image reconstruction system includes a memory encoding processor-executable routines for iteratively reconstructing an image and a processing component configured to access the memory and execute the processor-executable routines. The routines, when executed by the processing component, cause acts to be performed comprising: acquiring or accessing a set of projection data of a scanned region; iteratively performing a reconstruction operation to reconstruct an image of the region; as part of each reconstruction operation, applying a weight factor to each projection measurement, wherein the respective weight factors are determined based on both a respective projection measurement position and a reconstructed pixel location; and displaying or storing the image.

In an additional aspect of the present approach, one or more non-transitory computer-readable media encoding processor-executable routines are provided. In accordance with this aspect, the routines, when executed by a processor, cause acts to be performed comprising: acquiring or accessing a set of projection data of a scanned region; iteratively performing a reconstruction operation to reconstruct an image of the region; as part of each reconstruction operation, applying a weight factor to each projection measurement, wherein the respective weight factors are determined based on both a respective projection measurement position and a reconstructed pixel location; and displaying or storing the image.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a block diagram depicting components of a computed tomography (CT) imaging system, in accordance with aspect of the present disclosure;

FIG. 2 depicts algorithmic steps of a conventional iterative reconstruction, in accordance with aspect of the present disclosure;

FIG. 3 graphically depicts an example of an image region split into differing sub-region each reconstructed using different amounts of sinogram data, in accordance with aspect of the present disclosure;

FIG. 4 depicts algorithmic steps of an iterative reconstruction, in accordance with aspect of the present disclosure;

FIG. 5 depicts algorithmic steps of a further iterative reconstruction, in accordance with aspect of the present disclosure;

FIG. 6 depicts algorithmic steps of an additional iterative reconstruction, in accordance with aspect of the present disclosure;

FIG. 7 depicts an example of basis functions of sinogram weights, in accordance with aspect of the present disclosure;

FIG. 8 depicts an image generated using conventional iterative reconstruction; and

FIG. 9 depicts an image generated using an iterative reconstruction operation in accordance with aspect of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments. The term “pixel” is intended to include 2D pixels, 3D voxels, or in 4D or higher dimensional applications, the corresponding element of the reconstructed data.

One reconstruction technique used in CT imaging is iterative reconstruction. Use of iterative reconstruction techniques (in contrast to analytical methods) may be desirable for a variety of reasons, including image quality and/or reduced patient dose. As discussed herein, conventional iterative tomographic reconstruction (IR) uses statistical weights to modulate the importance of each sinogram ray for benefits in dose efficiency and image quality. However, because these weights are assigned in the sinogram-domain ray-by-ray, they apply globally to all image locations along the full length of the ray. That is, iterative reconstruction, is inherently “global” in that the whole object is modeled for reconstruction (regardless of the size of the clinical region of interest) to properly account for all absorption that contributes to the detector measurements in the forward model. Thus, individual pixel locations or sub-regions of the image cannot be differently weighted, such as based on spatial location, in conventional iterative reconstruction techniques.

In contrast, the present iterative reconstruction approach allows the use of varying sinogram weights in pixels or larger sub-regions in the reconstructed image. By way of example, the relative significance of each projection measurement may be determined based on both the measurement position and the location of the reconstructed pixel. Computationally, the significance of each projection based on these two factors is represented by a weight factor employed in the algorithmic computation. This approach can be applied to many CT imaging scenarios where flexible view-weighting in iterative reconstruction is needed. For example, this approach may be useful in wide-cone cardiac iterative reconstruction where conventional iterative reconstruction cannot achieve good temporal resolution while suppressing cone-beam artifacts at the same time. Further, the weights may vary over time in contexts where a time sequence of images are produced.

Thus, in such approaches, images may be reconstructed that have distinct regions which are each reconstructed using different weights, thereby improve the image quality of those regions relative to scenarios in which the same weighting is used across all regions. Likewise, to the extent that a region or regions may vary over time in a time-lapse or video context, different weighting may be applied in different regions of the image over time to improve image quality relative to conventional approaches where the same weighting is used throughout the image(s). Images and/or videos so produced can then be stored and/or displayed on a monitor, such as at the scan station or on a network-connected device, such as a workstation in a radiologist office. As regions of interest in the images may be presented with superior image quality relative to conventional images, such images and/or videos may improve the ability of a diagnostician to diagnose a condition and/or prescribe a treatment.

The approaches described herein may be suitable for use with a range of image reconstruction systems. However, to facilitate explanation, the present disclosure will primarily discuss the present reconstruction approaches in one particular context, that of a CT system. It should be understood that the following discussion may also be applicable to other image reconstruction modalities and systems as well as to non-medical contexts or any context where an image is reconstructed from projections or other forms of measurements.

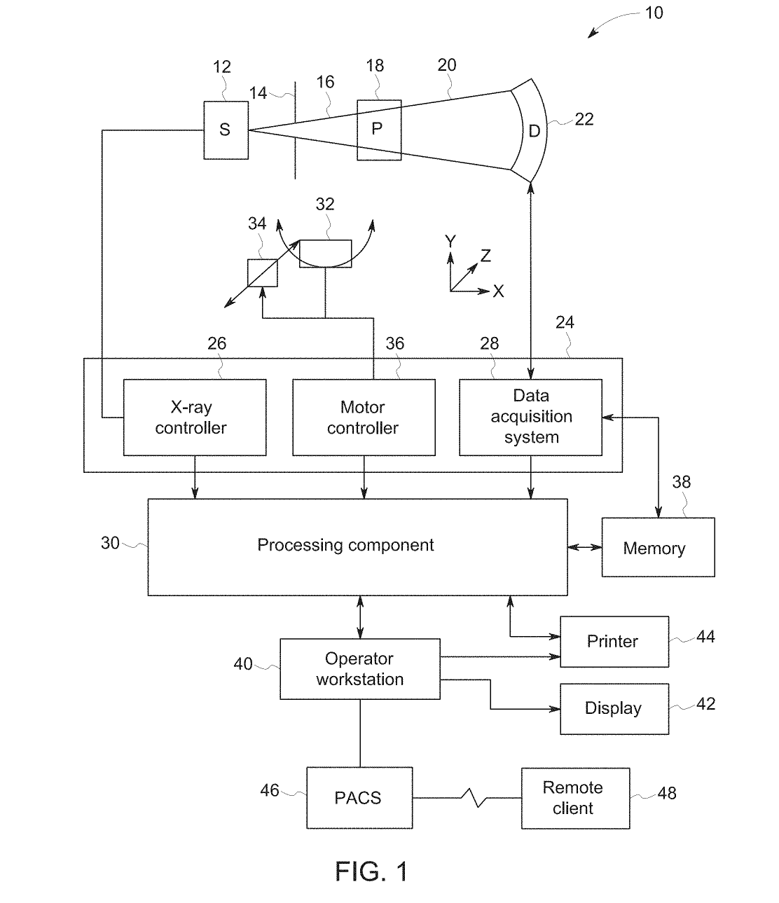

With this in mind, an example of a computer tomography (CT) imaging system 10 designed to acquire X-ray attenuation data at a variety of views around a patient (or other subject or object of interest) and suitable for spatially-adaptive sinogram weighting in iterative reconstruction is provided in FIG. 1. In the embodiment illustrated in FIG. 1, imaging system 10 includes a source of X-ray radiation 12 positioned adjacent to a collimator 14. The X-ray source 12 may be an X-ray tube, a distributed X-ray source (such as a solid-state or thermionic X-ray source) or any other source of X-ray radiation suitable for the acquisition of medical or other images.

The collimator 14 permits X-rays 16 to pass into a region in which a patient 18, is positioned. In the depicted example, the X-rays 16 are collimated to be a cone-shaped beam, i.e., a cone-beam that passes through the imaged volume. A portion of the X-ray radiation 20 passes through or around the patient 18 (or other subject of interest) and impacts a detector array, represented generally at reference numeral 22. Detector elements of the array produce electrical signals that represent the intensity of the incident X-rays 20. These signals are acquired and processed to reconstruct images of the features within the patient 18.

Source 12 is controlled by a system controller 24, which furnishes both power, and control signals for CT examination sequences, including acquisition of 2D localizer or scout images used to identify anatomy of interest within the patient for subsequent scan protocols. In the depicted embodiment, the system controller 24 controls the source 12 via an X-ray controller 26 which may be a component of the system controller 24. In such an embodiment, the X-ray controller 26 may be configured to provide power and timing signals to the X-ray source 12.

Moreover, the detector 22 is coupled to the system controller 24, which controls acquisition of the signals generated in the detector 22. In the depicted embodiment, the system controller 24 acquires the signals generated by the detector using a data acquisition system 28. The data acquisition system 28 receives data collected by readout electronics of the detector 22. The data acquisition system 28 may receive sampled analog signals from the detector 22 and convert the data to digital signals for subsequent processing by a processor 30 discussed below. Alternatively, in other embodiments the digital-to-analog conversion may be performed by circuitry provided on the detector 22 itself. The system controller 24 may also execute various signal processing and filtration functions with regard to the acquired image signals, such as for initial adjustment of dynamic ranges, interleaving of digital image data, and so forth.

In the embodiment illustrated in FIG. 1, system controller 24 is coupled to a rotational subsystem 32 and a linear positioning subsystem 34. The rotational subsystem 32 enables the X-ray source 12, collimator 14 and the detector 22 to be rotated one or multiple turns around the patient 18, such as rotated primarily in an x,y-plane about the patient. It should be noted that the rotational subsystem 32 might include a gantry upon which the respective X-ray emission and detection components are disposed. Thus, in such an embodiment, the system controller 24 may be utilized to operate the gantry.

The linear positioning subsystem 34 may enable the patient 18, or more specifically a table supporting the patient, to be displaced within the bore of the CT system 10, such as in the z-direction relative to rotation of the gantry. Thus, the table may be linearly moved (in a continuous or step-wise fashion) within the gantry to generate images of particular areas of the patient 18. In the depicted embodiment, the system controller 24 controls the movement of the rotational subsystem 32 and/or the linear positioning subsystem 34 via a motor controller 36.

In general, system controller 24 commands operation of the imaging system 10 (such as via the operation of the source 12, detector 22, and positioning systems described above) to execute examination protocols and to process acquired data. For example, the system controller 24, via the systems and controllers noted above, may rotate a gantry supporting the source 12 and detector 22 about a subject of interest so that X-ray attenuation data may be obtained at one or more views relative to the subject. In the present context, system controller 24 may also include signal processing circuitry, associated memory circuitry for storing programs and routines executed by the computer (such as routines for executing image reconstruction techniques employing differential weighting as described herein), as well as configuration parameters, image data, reconstructed images, and so forth.

In the depicted embodiment, the image signals acquired and processed by the system controller 24 are provided to a processing component 30 for reconstruction of images in accordance with the presently disclosed algorithms. The processing component 30 may be one or more general or application-specific microprocessors. The data collected by the data acquisition system 28 may be transmitted to the processing component 30 directly or after storage in a memory 38. Any type of memory suitable for storing data might be utilized by such an exemplary system 10. For example, the memory 38 may include one or more optical, magnetic, and/or solid-state memory storage structures. Moreover, the memory 38 may be located at the acquisition system site and/or may include remote storage devices for storing data, processing parameters, and/or routines for image reconstruction as described herein.

The processing component 30 may be configured to receive commands and scanning parameters from an operator via an operator workstation 40, typically equipped with a keyboard and/or other input devices. An operator may control the system 10 via the operator workstation 40. Thus, the operator may observe the reconstructed images and/or otherwise operate the system 10 using the operator workstation 40. For example, a display 42 coupled to the operator workstation 40 may be utilized to observe the reconstructed images and to control imaging. Additionally, the images may also be printed by a printer 44 which may be coupled to the operator workstation 40.

Further, the processing component 30 and operator workstation 40 may be coupled to other output devices, which may include standard or special purpose computer monitors and associated processing circuitry. One or more operator workstations 40 may be further linked in the system for outputting system parameters, requesting examinations, viewing images, and so forth. In general, displays, printers, workstations, and similar devices supplied within the system may be local to the data acquisition components, or may be remote from these components, such as elsewhere within an institution or hospital, or in an entirely different location, linked to the image acquisition system via one or more configurable networks, such as the Internet, virtual private networks, and so forth.

It should be further noted that the operator workstation 40 may also be coupled to a picture archiving and communications system (PACS) 46. PACS 46 may in turn be coupled to a remote client 48, radiology department information system (RIS), hospital information system (HIS) or to an internal or external network, so that others at different locations may gain access to the raw or processed image data.

While the preceding discussion has treated the various exemplary components of the imaging system 10 separately, these various components may be provided within a common platform or in interconnected platforms. For example, the processing component 30, memory 38, and operator workstation 40 may be provided collectively as a general or special purpose computer or workstation configured to operate in accordance with the aspects of the present disclosure. In such embodiments, the general or special purpose computer may be provided as a separate component with respect to the data acquisition components of the system 10 or may be provided in a common platform with such components. Likewise, the system controller 24 may be provided as part of such a computer or workstation or as part of a separate system dedicated to image acquisition.

As discussed herein, the system 10 of FIG. 1 may be used to conduct a computed tomography (CT) scan by measuring a series of projection images from many different angles around a patient 18 or object. The projection images acquired at different view angles can be combined into a sinogram, which collects the multiple views into a single data set. A reconstruction algorithm processes the sinogram to produce a space-domain image representing the patient 18 or object.

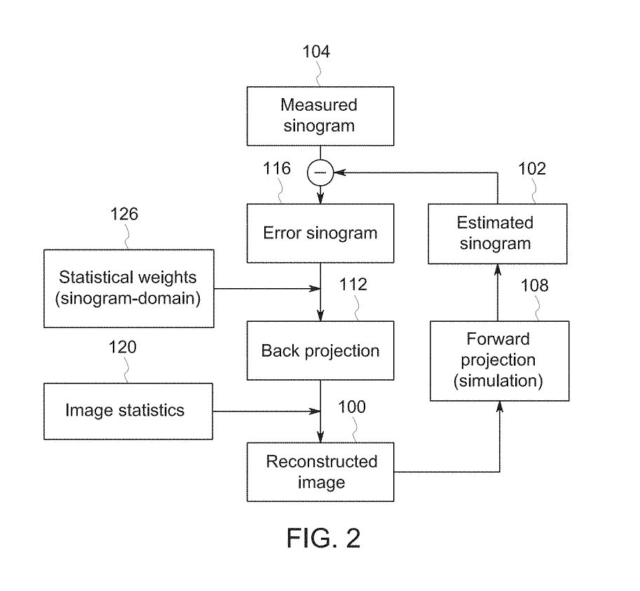

There are multiple methods for image reconstruction. For example, iterative reconstruction techniques are used to produce images of high quality while reducing the required radiation dose. An example of a conventional iterative reconstruction approach is depicted in FIG. 2 as a block diagram showing a simplified representation of an iterative reconstruction algorithm. The objective of such an iterative reconstruction approach is to produce the reconstructed images 100 that would result in estimated sinograms 102 that best match the set of measured sinograms 104 collected from a CT scan. On each iteration of the algorithm, a forward model 108 takes the geometry and other characteristics of the CT system, and computes the estimated sinogram 102 that would be produced by the current reconstructed image estimate 100 of the unknown object or patient. The forward model 108 is essentially simulating the attenuation of X-rays as they pass from the X-ray source, through the patient and into the detector of the CT system based on the current reconstructed image estimate. The estimated sinogram 102 is compared against the measured sinogram 104 from the CT scan. In the depicted example, the comparison of the estimated sinogram 102 and the measured sinogram 104 takes the form of determining a difference, i.e., error sinogram 116, such as by subtracting the estimated sinogram 102 from the measured sinogram 104. The significance of the error sinogram at different positions can be further weighted by the weighting factors 126. Based on this weighted comparison, a backprojection 112 is calculated. The backprojection can further incorporate image statistics 120 so that the generated updated reconstructed estimate 100 conforms to or approaches the desired regularity condition of the scanned subject. The image statistics 120 may be based on a regularization function or prior distribution function image statistics and may reflect desired properties of the reconstructed image in different locations within the image and under various scan conditions.

With respect to the backprojection step 112, in conventional iterative reconstruction approaches the statistical weights 126 are determined on a ray-by-ray basis, to reflect noise levels of each sinogram measurement. The statistical weights 126 are inherently “global” for all pixel locations along the full length of the ray, which does not allow flexible adjustments of the weights as a function of pixel locations in the image domain.

This can be understood from the aspect that iterative reconstruction is typically based on a single cost function of all image pixel variables. Iterations are performed of the algorithm until the cost function reaches a specified threshold, such as being minimized. As a simplified example, consider iterative reconstruction based on the weighted least-squares cost function:

$$\hat{x} = {{argmin}_{x} ( y - Ax )}^{T} W ( y - Ax ) \qquad ( 1 )$$

The statistical weights, denoted by the matrix W, have the same dimension as the sinogram y, while x is the image to be estimated and A is the system matrix. In a conventional approach, there is no room to adjust the weights for different sub-regions of the image x.

This unavailability of spatially-dependent sinogram-weighting or view-weighting in iterative reconstruction techniques is in contrast to its availability in analytical reconstruction approaches, such as filtered backprojection (FBP). In these analytical approaches, the reconstruction at each spatial location can be carried out by a closed form equation and the reconstructions at different spatial locations may be carried out with computations that are independent of each other. Thus, differential weighting may be applied.

However, iterative reconstruction is based on minimizing a cost function that considers all pixel jointly is intrinsically “global”, and thus not suitable in conventional approaches for differential weighting of the sinogram measurements in sub-regions or pixels in the reconstructed image. This may prove problematic, however, in certain CT imaging scenarios that would benefit from spatially-adaptive weights to optimize image quality and reduce various artifacts. For example, in a wide-cone cardiac CT context, the coronary arteries are subject to motion and it may be useful to use a relatively small portion of the acquired data, such as masked by a Parker window, to reduce the inconsistency in the sinogram data and reduce motion artifacts in the reconstructed image. However, in non-cardiac regions of the same scan that are not subject to motion, such as the spine and abdomen, it may instead be desirable to use all available data to maximize dose efficiency and reduce cone beam artifacts.

Another example where spatially-adaptive weights may be useful in in the helical CT context in which the patient is linearly displaced in the image bore while the gantry rotates. Due to small inconsistencies caused by patient motion and/or system inaccuracy, pin wheel (hurricane, HAR) artifacts may be present in the reconstructed image if all sinogram data are used in the iterative reconstruction process. To reduce the pin wheel artifacts, it may be desirable to use less than the full data in image regions that are prone to pin wheel artifacts. However, in regions where the pin wheel artifacts are less noticeable, such as the center of the image, it may be desirable to use the full data to maximize dose efficiency.

In a further example, spatially-adaptive weighting may be useful in various other contexts that may lead to artifacts or deficiencies in the image that may be spatially localized. For example, spatially-adaptive weighting may be useful to address localized issues related to scatter, low-signal, metal artifacts (such as due to metal implants or device in the scan area) and so forth. In such contexts, by using spatially-adaptive weighting, some X-rays could be weighted less or more in certain image sub-regions of the image.

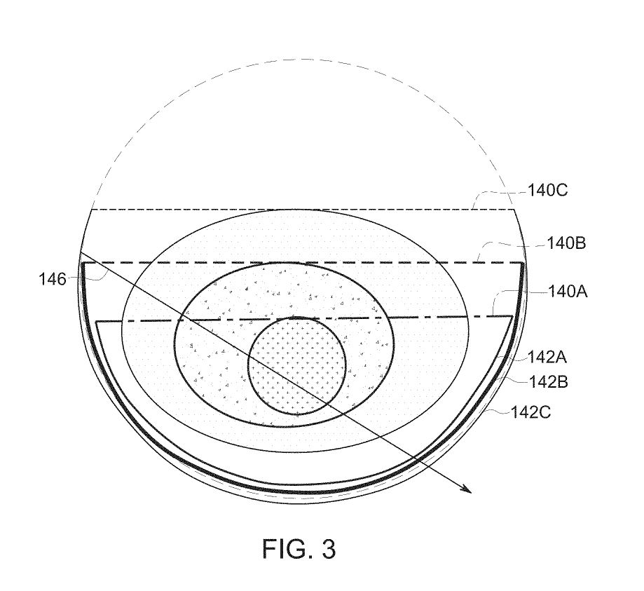

With the preceding in mind, the present iterative reconstruction approach employs spatially-adaptive sinogram weighting. This weighting may take various forms. For example, in one implementation, the weight factors may be determined by applying a pixel-dependent multiplicative factor to a conventional pixel-independent weight. In a further implementation, the weight factors are determined by a pixel-dependent temporal window function (such as is illustrated graphically in FIG. 3). In another embodiment discussed herein, the weight factors may be determined by a linear combination of basis temporal window functions.

Turning to FIG. 3, an example is illustrated where different image regions 140A, 140B, 140C are reconstructed with different amounts of sinogram data. As used herein, the different image regions 140 may, in certain embodiments, be based on or derived as temporal window functions that are pixel-dependent. That is, the reconstructed image may be segmented into multiple (i.e., two or more) sub-image regions that each use a different set of temporal windows. For example, in a chest image, half-scan weights may be applied in a cardiac region (to reduce artifacts due to motion in this region) and full-scan weights may be applied in a background region where little motion is expected. In such an approach, the temporal window(s) may be determined based on a first and last view index (such as may be associated with one of the arcs 142 discussed below).

The arcs 142A, 142B, 142C of different lengths represent the portion of the X-ray source trajectory, and the corresponding sinogram data, that are weighted differently from one another based on corresponding image regions 140. Among the three arcs of the X-ray source trajectory, the shortest one 142A is used for the reconstruction of the inner-most sub-region, whereas the longest one 142C is used for the reconstruction of the outer-most sub-region. This contrasts with the conventional iterative reconstruction, where different image regions would have the same sinogram weights. In this example, the projection measurement along the ray 146 is used for reconstruction in the sub-region 140C, but not in sub-regions 140B and 140A.

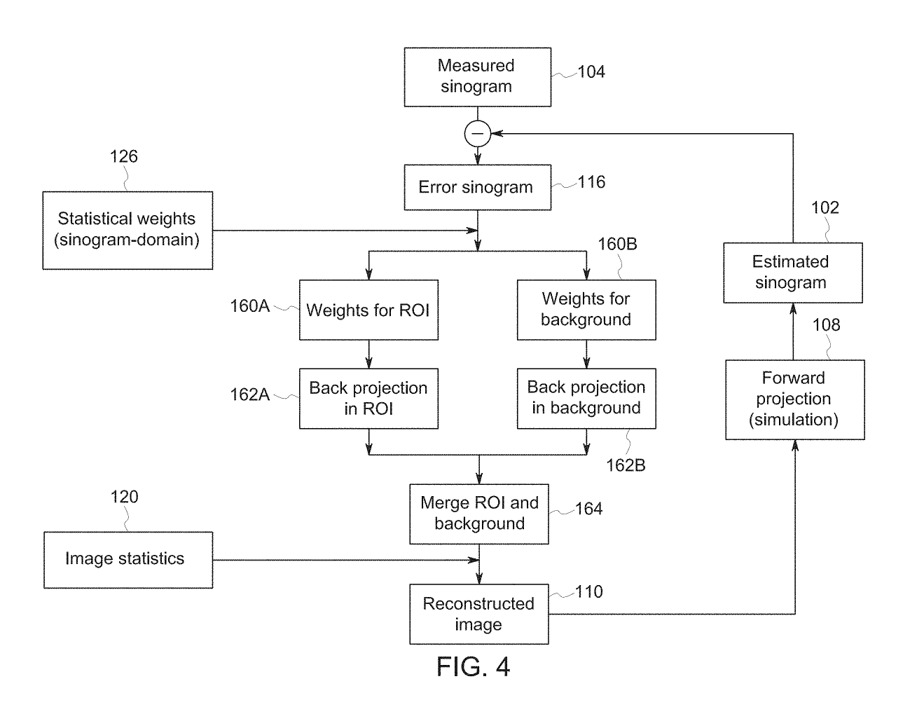

Turning to FIG. 4, a flow-type diagram is provided of one implementation of the present iterative reconstruction approach with multiple backprojectors 162, here a region-of-interest (ROI) backprojector 162A and a background backprojector 162B, employing different statistical weights 160, here ROI weights 160A and background weights 160B. Thus, in this implementation, instead of using a single back projector as shown in FIG. 2, multiple backprojectors 162 are instead employed, each associated with a different set of sinogram weights 160. In this manner, different weights 160 may be used in different image sub-regions, such as a region-of-interest and a background region. In this implementation, the backprojection only needs to be computed in the corresponding sub-region, therefore the computational cost would be less than performing the backprojection operation in the entire reconstructed image. The different sub-image regions after the separate backprojection operations can be merged (block 164) by image-domain masks to generate the current reconstructed image estimate 110. Smoothing of the mask boundaries can be employed for a smooth transition between the sub-regions.

Consistent with FIG. 4, the merged backprojection 164 is:

$${g}_{j} = \{ \begin{matrix}{\sum }_{i} {a}_{ij} {v}_{i}^{ROI} {e}_{i} & j ∈ ROI \\ {\sum }_{i} {a}_{ij} {v}_{i}^{{BKGND}_{{e}_{i}}} & otherwise\end{matrix} \qquad ( 2 )$$

where eᵢ=wᵢ(yᵢ−[Ax]ᵢ) denotes the conventionally weighted error sinogram at detector cell i, with wᵢ denoting the conventional sinogram-domain weight that typically reflect noise levels of measurement at cell i, and vᵢ^{ROI} and vᵢ^{BKGND} denote the weights for cell i in the ROI and the background, respectively. The weights vᵢ^{ROI} and vᵢ^{BKGND} can be designed independent of each other to optimize reconstruction quality.

In one embodiment of this iterative reconstruction approach, and the other iterative reconstruction approaches discussed herein, numerical optimization techniques such as a line search may be employed to ensures monotonic decrease of the cost function after each iteration until a specified threshold, such as cost function minimization, is reached. In addition, in these and other embodiments, a relaxation factor may be employed to improve convergence. Further, with respect to this and other embodiments, the statistically-weighted, backprojected error may be spatially filtered. Furthermore, with respect to this and other embodiments, ordered subsets, conjugate gradient, preconditioner, Nesterov's optimal gradient iteration, method of momentum, and other numerical optimization techniques can be used to improve the speed of convergence and reduce the computational overhead.

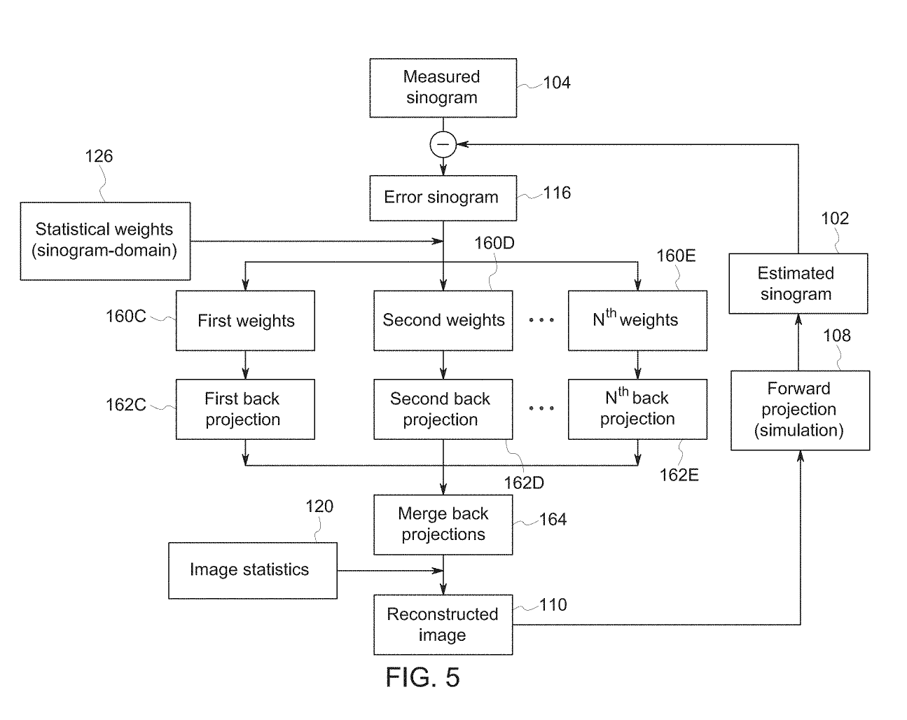

Turning to FIG. 5, it is illustrated that this implementation can be extended beyond two sub-regions to N sub-regions, with a different backprojector 162 (e.g., backprojectors 162C, 162D, 162E, and so forth) and separate weights 160 (e.g., weights 160C, 160D, 160E, and so forth) provided for each sub-region.

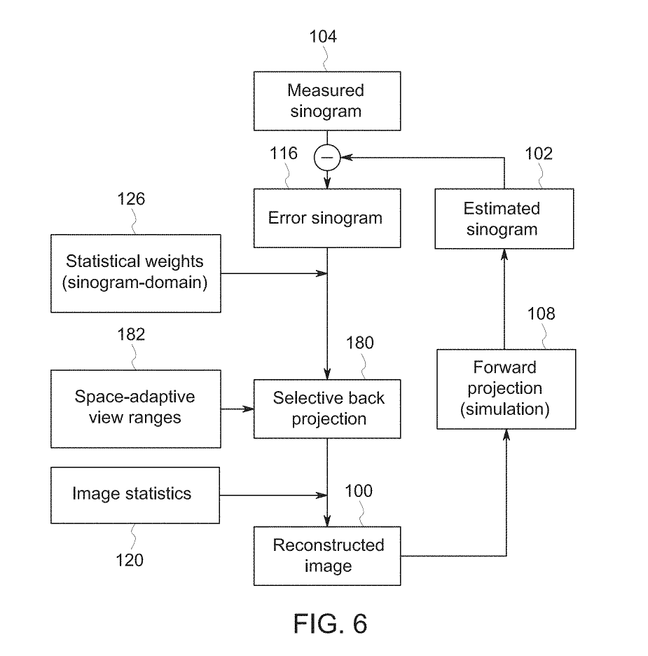

In the preceding implementation, each iteration or the algorithm employs multiple backprojection operations, which may incur computational overhead compared to the conventional approach shown in FIG. 2 where only a single backprojection is used each iteration. Conversely, turning to FIG. 6, in a different implementation the computational overhead may be reduced in the case where the space-dependent sinogram weights, e.g., vⱼᵢ, are binary (i.e., one or zero). In this circumstance, the multiple back projections can instead be implemented as a single selective backprojection operation 180 that allows the selection of different view ranges at different pixel locations (e.g., spatially-adaptive view ranges 182). For example, the backprojection operation at a given pixel location skips measurements (i.e., is selective) that are outside the temporal window function at the respective pixel location. The selective backprojection is defined as:

$${g}_{j} = {\sum }_{i ∈ {S}_{j}} {a}_{ij} {e}_{i} \qquad ( 3 )$$

where Sⱼ denotes the set of sinogram bins that should be used for pixel j. For each pixel, the backprojection operation restricts the range of summation to rays that belong to the set Sⱼ, and a single backprojection operation 180 suffices. Because Sⱼ can vary across j, it becomes a way to realize space-variant sinogram weights in iterative reconstruction.

With the preceding in mind, the prior implementations may be generalized to a more flexible, spatially-adaptive backprojection, defined as:

$${g}_{j} = {\sum }_{i} {a}_{ij} {v}_{ij} {e}_{i} \qquad ( 4 )$$

where the coefficient vᵢⱼ denotes a weight factor that depends on both sinogram and image locations.

Although this is a generalized form of sinogram weight, the computational cost for explicit generation and storage of vᵢⱼ may be prohibitive. With this in mind, in a further embodiment, the weight vᵢⱼ may be expressed as the linear combination of a small number of basis functions that can be computed on-the-fly as:

$${v}_{ij} = {\sum }_{k = 1}^{K} {α}_{ij} {v}_{ki} \\ ⇒ {g}_{j} = {\sum }_{k = 1}^{K} {α}_{jk} {\sum }_{i} {a}_{ij} {v}_{ki} {e}_{i} = {\sum }_{i} [ {a}_{ij} ( {\sum }_{k = 1}^{K} {α}_{jk} {v}_{ki} ) {e}_{i} ] \qquad ( 5 )$$

where vₖᵢ denotes the kth basis function of the weights; αⱼₖ denotes the coefficient of the kth basis function at pixel j; K denotes the number of basis functions. Typically, K is less than the number of sinogram bins, therefore this factorized form can reduce the overhead to explicitly store the full weights vᵢⱼ.

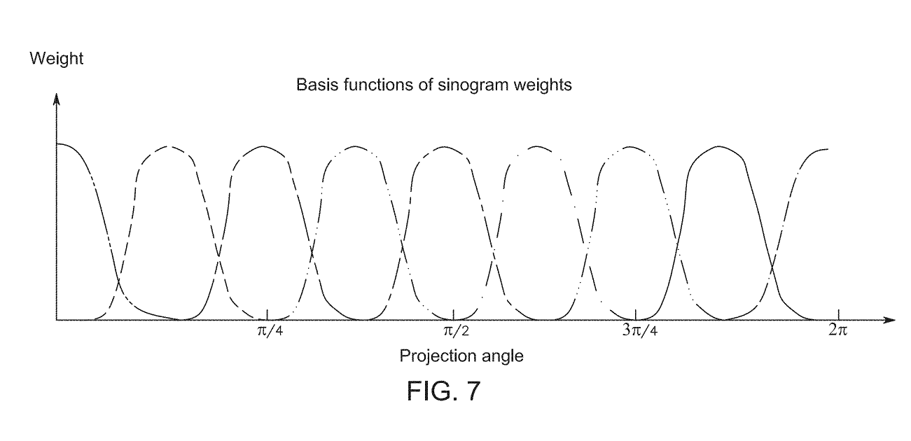

FIG. 7 shows an example where each basis function represents a sinogram-domain window function that covers a narrow range of projection angles. Eight basis functions are shown, each corresponding to an angular range of π/8. A linear combination of these basis functions can form various sinogram weights. In particular, by a linear combination of these basis function, a view-range window can be generated at each image pixel location j. Should higher precision of these window function be needed, the number of basis functions, K, can be increased.

With respect to implementation, a study was conducted in which iterative reconstruction was implemented with spatially-adaptive sinogram weighting using multiple backprojectors having different respective sinogram weights. The approach was applied to wide-cone cardiac CT, with a trans-axial coverage of 16 cm. To improve the tradeoff between temporal resolution and cone-beam artifacts, short-scan sinogram weights were used for the heart region of the patient, while full-scan sinogram weights were used for the rest of the patient to reduce missing data in regions with high cone angles. Qualitative and quantitative evaluations of the resulting reconstructed images showed that the present approach achieved the same temporal resolution as a conventional short-scan, but prevented or limited artifacts in non-heart regions and maintained the consistent image quality in these regions as a conventional full-scan.

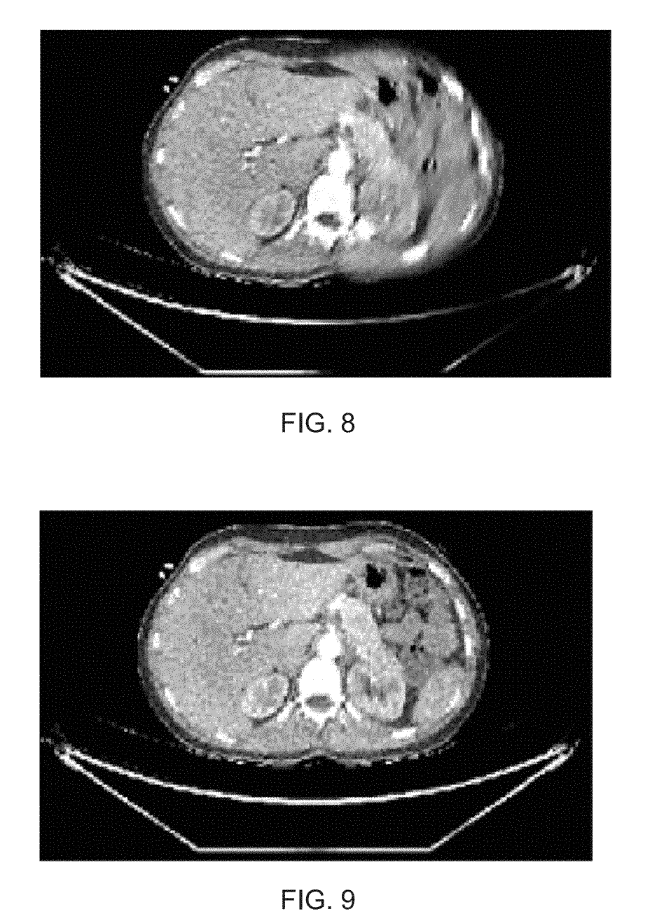

Results of this approach and a conventional iterative reconstruction are shown in FIGS. 8 and 9, with each figures respectively showing reconstructed images in edge slices with conventional sinogram weighting (FIG. 8) and adaptive sinogram weighting as discussed herein (FIG. 9). In edge slices, the conventionally reconstructed image shown in FIG. 8 suffers from artifacts caused by missing data due to the short-scan weights, but the image reconstructed using spatially adaptive sinogram weighting does not exhibit the same artifacts. As may be appreciated, such artifact-reduced or artifact-free images may be stored and/or displayed (such as on the scanner itself or on a PACS system for remote or future viewing) and used in making diagnosis or treatment decisions by a reviewer.

Technical effects of the invention include the use of differing or varying sinogram weights in pixels or larger sub-regions in the reconstructed image. In certain implementations, this may take the form of applying spatially-adaptive sinogram weighting during an iterative reconstruction process to improve image quality.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A tomographic iterative reconstruction method, comprising:
acquiring or accessing a set of projection data of a scanned region;
iteratively performing a reconstruction operation to reconstruct an image of the region;
as part of each reconstruction operation, applying a weight factor to each projection measurement, wherein the respective weight factors are determined based on both a respective projection measurement position and a reconstructed pixel location; and
displaying or storing the image.

2. The tomographic iterative reconstruction method of claim 1, wherein the reconstruction operation is based on descent of a cost function.

3. The tomographic iterative reconstruction method of claim 1, wherein each weight factor corresponds to a relative significance of the corresponding projection measurements to the image or a sub-region of the image.

4. The tomographic iterative reconstruction method of claim 1, wherein weight factors are incorporated into a backprojection operation.

5. The tomographic iterative reconstruction method of claim 1, wherein the weight factors are determined by applying a pixel-dependent multiplicative factor to a pixel-independent weight.

6. The tomographic iterative reconstruction method of claim 1, further comprising performing one or more numerical optimization techniques each iteration to ensure monotonic decrease of a cost function during each iteration.

7. The tomographic iterative reconstruction method of claim 4, wherein the numerical optimization techniques comprise one or more of a line search or a relaxation factor.

8. The tomographic iterative reconstruction method of claim 1, further comprising performing numerical optimization techniques to improve the speed of convergence and reduce the computational overhead.

9. The tomographic iterative reconstruction method of claim 8, wherein the numerical optimization techniques comprise one or more of ordered subsets, conjugate gradient, preconditioner, Nesterov's optimal gradient iteration, or method of momentum.

10. The tomographic iterative reconstruction method of claim 1, further comprising spatially filtering a weighted backprojection error generated each iteration.

11. The tomographic iterative reconstruction method of claim 1, wherein the weight factors are determined based on a pixel-dependent temporal window function.

12. The tomographic iterative reconstruction method of claim 11, wherein the image is segmented into multiple sub-image regions that are each subject to a different set of temporal windows.

13. The tomographic iterative reconstruction method of claim 11, wherein each temporal window is determined by its first and last view index.

14. The tomographic iterative reconstruction method of claim 11, wherein the weight factors are determined by a linear combination of basis temporal window functions.

15. The tomographic iterative reconstruction method of claim 12, wherein the backprojection operation at a pixel location skips measurements that are outside the temporal window function at the respective pixel location.

16. The tomographic iterative reconstruction method of claim 1, wherein the weight factors are determined by spatial frequency relationships between the projection-domain and image-domain.

17. The tomographic iterative reconstruction method of claim 1, wherein the weight factors varies with time.

18. An image reconstruction system, comprising:
a memory encoding processor-executable routines for iteratively reconstructing an image;
a processing component configured to access the memory and execute the processor-executable routines, wherein the routines, when executed by the processing component, cause acts to be performed comprising:
acquiring or accessing a set of projection data of a scanned region;
iteratively performing a reconstruction operation to reconstruct an image of the region;
as part of each reconstruction operation, applying a weight factor to each projection measurement, wherein the respective weight factors are determined based on both a respective projection measurement position and a reconstructed pixel location; and
displaying or storing the image.

19. The image reconstruction system of claim 18, wherein each weight factor corresponds to a relative significance of the corresponding projection measurements to the image or a sub-region of the image.

20. The image reconstruction system of claim 18, wherein the weight factors are determined based on a pixel-dependent temporal window function.

21. One or more non-transitory computer-readable media encoding processor-executable routines, wherein the routines, when executed by a processor, cause acts to be performed comprising:
acquiring or accessing a set of projection data of a scanned region;
iteratively performing a reconstruction operation to reconstruct an image of the region;
as part of each reconstruction operation, applying a weight factor to each projection measurement, wherein the respective weight factors are determined based on both a respective projection measurement position and a reconstructed pixel location; and
displaying or storing the image.
